(19) United States
(12) Patent Application Publication
(10) Pub. No.: US 2013/0278740 A1
(43) Pub. Date: Oct. 24, 2013

(54) IMAGING SYSTEM AND METHOD USING MULTICORE FIBER

(71) Applicants: Zeev Zalevsky, Rosh HaAyin (IL); Asaf Shahmoon, Petach Tikva (IL); Hamutal Slovin, Moshav Galia (IL)

(72) Inventors: Zeev Zalevsky, Rosh HaAyin (IL); Asaf Shahmoon, Petach Tikva (IL); Hamutal Slovin, Moshav Galia (IL)

(73) Assignee: BAR ILAN UNIVERSITY, Ramat Gan (IL)

(21) Appl. No.: 13/978,423

(22) PCT Filed: Jan. 5, 2012
(86) PCT No.: PCT/IL2012/050004

§ 371 Date: Jul. 5, 2013

Related U.S. Application Data
(60) Provisional application No. 61/457,116, filed on Jan. 5, 2011.

(51) Int. Cl. A61B 1/07

(52) U.S. Cl. CPC A61B 1/07
USPC 348/76

(57) ABSTRACT

The present invention provides a system for imaging an object comprising: an optical imaging unit for imaging an object on a detection array, the optical imaging unit defining an optical axis and comprising a multicore fiber configured to collect light from the object at an input edge of the multicore fiber and transfer collected light to an output edge of the multicore fiber; a displacing unit configured to shift the input edge of the multicore fiber relatively to the object in a plane substantially perpendicular to the optical axis to obtain a set of shifted images of the object; and an operating unit configured to operate the displacing unit by setting a shifting amplitude to either a first amplitude inferior or equal to the diameter of a core of the multicore fiber or a second amplitude superior or equal to the diameter of the multicore fiber.

### FIELD OF THE INVENTION

The present invention relates generally to the domain of imaging systems and methods.

### REFERENCES

The following references belong to the technical background of the present invention:
[1] G. Unfriedl, F. Wieser, A. Albrecht, A. Kaider and F. Nagele, “Flexible versus rigid endoscopes for outpatient hysteroscopy: a prospective randomized clinical trial” Human Reproduction 16, 168-171 (2001).
[2] R. P. J. Barretto, B. Messerschmidt and M. J. Schnitzer, “In vivo fluorescence imaging with high-resolution microlenses,” Nature methods 6, 511-514 (2009).
[3] B. A. Flusberg, E. D. Cocker, W. Piyawattanametha, J. C. Jung, E. L. M. Cheung and M. J. Schnitzer, “Fiber-optic fluorescence imaging,” Nature methods 2, 941-950 (2005).
[4] M. E. Llewellyn, R. P. J. Barretto, S. L. Delp and M. J. Schnitzer, “Minimally invasive high-speed imaging of sarcomere contractile dynamics in mice and humans,” Nature 454, 784-788 (2008).
[5] K. Deisseroth, G. Feng, A. K. Majewska, G. Miesenbock, A. Ting and M. J. Schnitzer, “Next-Generation Optical Technologies for Illuminating Genetically Targeted Brain Circuits,” The Journal of Neuroscience, 26, 10380-10386 (2006).
[6] B. A. Flusberg, J. C. Jung, E. D. Cocker, E. P. Anderson and M. J. Schnitzer, “In vivo brain imaging using a portable 3.9 gram two-photon fluorescence microendoscope,” Opt. Lett. 30, 2272-2274 (2005).
[7] W. Piyawattanametha, E. D. Cocker, L. D. Burns, R. P. J. Barretto, J. C. Jung, H. Ra, O. Solgaard and M. J. Schnitzer, “In vivo brain imaging using a portable 2.9 g two-photon microscope based on a microelectromechanical systems scanning mirror,” Opt. Lett. 34, 2309-2311 (2009).
[8] Z. Zalevsky and D. Mendlovic, Optical Super Resolution, Springer (2004).
[9] A. Borkowski, Z. Zalevsky and B. Javidi, “Geometrical Super Resolved Imaging Using Non periodic Spatial Masking,” JOSA A 26, 589-601 (2009).
[10] J. Fortin, P. Chevrette, and R. Plante “Evaluation of the microscanning process,” SPIE Vol. 2269, Infrared Technology XX, Bjorn F. Andresen, Editors, 271-279 (1994).
[11] V. Mico, Z. Zalevsky and J. Garcia, “Common-path Phase-shifting Digital Holographic Microscopy: a way to Quantitative Phase Imaging and Superresolution,” Opt. Commun. 281, 4273-4281 (2008).

### BACKGROUND OF THE INVENTION

Endoscopes are the common medical instrumentation to perform medical inspection of internal organs. There are two main types of endoscopes: flexible and rigid. The flexible endoscopes are being constructed out of a bundle of single mode fibers while each fiber in the bundle transmits backwards spatial information corresponding to a single spatial point, i.e. a single pixel. The fibers bundle may go into the body while the imaging camera is located outside. Interface optics adapts the photonic information coming out of the bundle to the detection camera. The reason for using single mode fiber for each fiber in the bundle rather than multi mode fibers (capable of transmitting spatial information that is corresponding to plurality of pixels) is related to the fact that when inserting the endoscope and while navigating it inside the body it may be bent. When multi mode fibers are bent the spatial modes are coupled to each other and the image is strongly distorted. The typical diameter of a single mode fiber in the bundle is about 30 μm (this is the diameter of its cladding, the core has diameter of about 8-9 μm). The typical number of fibers in the bundle is about 10,000-30,000. Typical overall diameter (of the entire bundle) is about 3 mm-5 mm.

Another type of endoscopes is called rigid endoscope. In this case the camera going inside the body of the patient rather than staying outside while it is located on the edge of a rigid stick. Although image quality of rigid endoscopes is usually better and they allow not only backwards transmission of images but also other medical treatment procedures, their main disadvantage is related to the fact that they are indeed rigid and thus less flexible and less adapted for in-body navigation procedures.

There are alternative solutions to endoscopy which for instance involve pills swallowed by the patient and capable of capturing images of internal organs while propagated through the stomach and the intestine.

Multi core fibers were also proven to be suitable to perform high quality imaging tasks. In Refs. [2-4] one may see an overview of the state of the art of in vivo fluorescence imaging with high resolution micro lenses. In Refs. [5-7] one may see the demonstration of this micro endoscope for in vivo brain fluorescence imaging application. The use of multicore fibers might be preferred in invasive applications as it minimizes damage due to the small diameter of such an instrument.

For example, an endoscope utilizing a multicore fiber is described in US Patent Application US-A1-2010/0046897 which discloses an endoscope system including an image fiber with an image fiber main body made of a plurality of cores for forming pixels and a cladding common thereto; and an optical system connected to an eyepiece side of the image fiber for causing laser light to enter the image fiber and for taking in an image from the image fiber, in which the image fiber has the cores arranged substantially uniformly over a cross-section of the image fiber main body, the cross-section being perpendicular to a longitudinal direction of the image fiber main body.

### GENERAL DESCRIPTION

The use of multicore fibers is advantageous in various applications, including medical applications, because of their small size and a possibility of making the instrument desirably flexible, if needed. However, multicore fiber based imaging faces issues related to limited resolution and/or field of view when used for imaging in near field or in far field conditions.

The present invention provides with a novel imaging system and method enabling to overcome these limitations. The imaging system of the invention includes a multicore fiber forming or being part of an optical imaging unit for imaging an object on a detection array. The multicore fiber by its input edge faces an object plane and by its output edge faces the detector plane. The multicore fiber thus collects light from the object at the input edge and transfers collected light to the output edge. Further provided in the imaging system is a displacing unit which operates to provide at least a lateral shift of the input edge of the multicore fiber relatively to the object, i.e. in a plane substantially perpendicular to the optical axis of the optical imaging unit. By this, a set of shifted images of the object can be sequentially obtained at the detector array. The displacing unit is controllably operated by an operating unit which sets a shifting amplitude to be either a first amplitude inferior or equal to the diameter of a core in the multicore fiber or a second amplitude superior or equal to the diameter of the multicore fiber.

As will be described further below, when imaging using a near field mode, the spatial resolution might be sufficiently high, however, the field of view is generally limited. By laterally shifting the multicore fiber with the second shifting amplitude, the field of view in the combined image formed by multiple successively acquired images, can be improved. On the other hand, when imaging an object using a far field mode, the field of view might be sufficient but the spatial resolution is typically limited. This can be solved, or at least partially solved, by using the lateral shift of the multicore fiber with the first shifting amplitude.

The invention provides for utilizing both the lateral shift of the multicore fiber, as described above, and also a longitudinal shift of the system operation between the far and near field modes. The latter can be implemented by either shifting the input edge of the multicore fiber itself, or alternatively (or additionally) using an imaging optics movable along the optical axis of the optical imaging unit. Practically, it would be easier to locate such a lens between the multicore fiber and the detector array, but generally a movable lens may be between the object and the multicore fiber.

Thus, according to one broad aspect of the present invention, there is provided a system for imaging an object comprising an optical imaging unit defining an optical axis and for imaging an object on a detection array, the optical imaging unit comprising a multicore fiber configured to collect light from the object at an input edge of the multicore fiber and transfer collected light to an output edge of the multicore fiber; a displacing unit configured to shift the input edge of the multicore fiber relatively to the object in a plane substantially perpendicular to the optical axis to obtain a set of shifted images of the object; and an operating unit configured to operate the displacing unit by setting a shifting amplitude to either a first amplitude inferior or equal to the diameter of a core of the multicore fiber or a second amplitude superior or equal to the diameter of the multicore fiber.

In some embodiment, the optical imaging unit comprises an optical assembly configured to collect light from the output edge of the multicore fiber and form an image of the object on the detection array.

In some embodiment, the optical imaging unit is configured for selectively operating in either one of a near field and far field imaging modes.

In some embodiment, the optical imaging unit comprises a lens unit arranged upstream of the input edge of the multicore fiber with respect to a direction of light propagation through the system, said lens unit being displaceable along the optical axis with respect to the object.

In some embodiment, the system further comprises a processing unit connectable to the detection array and configured to receive and process data indicative of the set of shifted images for obtaining a combined image of the object by interlacing one or more of said shifted images, said combined image having the improved resolution and/or field of view.

In some embodiment, the system further comprises a detection unit configured to monitor a distance between the object and the input edge of the multicore fiber to thereby enable the operation of the optical imaging unit in either one of the near field and far field imaging modes.

In some embodiment, the system further comprises a display unit for displaying at least one of the images.

In some embodiment, the operating unit comprises an input utility configured to receive input from a user defining whether the field of view or resolution of the imaging is to be improved.

In some embodiments, the operating unit further comprises a communication utility to communicate with the detection unit, and a shift controller for setting the shifting amplitude based on a distance between the object and the input edge of the multicore fiber and on whether a field of view or a resolution of the original image is to be improved.

In some embodiment, the operating unit comprises a communication utility to communicate with the input unit.

In some embodiment, the multicore fiber is either a fiber bundle or a photonic crystal.

In some embodiment, the multicore fiber has a polygonal cross section defining two opposite substantially parallel facets.

In some embodiment, the cross section of the multicore fiber is rectangular.

In some embodiment, the system further comprises electrodes located on said opposite facets of the multicore fiber to carry out at least one of electrical stimulation and sensing temperature using Peltier effect.

In some embodiment, the system further comprises a coherent light source configured to illuminate the object and provide a reference wave front; and an holographic or interferometric setup configured to provide interference between the reference wave front and a reflected wave front reflected by the object and transferred by the multicore fiber, thereby providing phase information on the light reflected by the object.

According to another broad aspect of the invention, there is provided a method for imaging an object comprising: transferring light coming from the object through a multicore fiber having an input edge and an output edge; imaging the object on a detection array by collecting light from the output edge of the multicore fiber; setting a shifting amplitude for multicore fiber, such that the shifting amplitude is either a first amplitude inferior or equal to the diameter of a core of the multicore fiber or a second amplitude superior or equal to the diameter of the multicore fiber, to enable improvement of either resolution or field of view of imaging; shifting the input edge of the multicore fiber using said shifting amplitude, thereby obtaining a set of shifted images of the object; and processing said set of shifted images in order to obtain a combined image of the object by interlacing said shifted images for improving the resolution or field of view.

In some embodiment, the method further comprises selectively imaging the object in either one of a near field and far field imaging modes.

In some embodiment, the method comprises moving a lens unit, in front of the input edge of the multicore fiber, along said axis of light propagation, with respect to the object.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1 is a general diagram illustrating a system for imaging an object according to an embodiment of the present invention.

FIG. 2 is a general diagram illustrating a method for imaging an object according to an embodiment of the present invention.

FIG. 3 is a picture of a probe of a system according to an embodiment of the present invention.

FIG. 4 is an image of an output edge of a probe of a system imaging an object according to an embodiment of the present invention.

FIGS. 5A-5B illustrate experimental results of images acquired by an imaging system according to an embodiment of the present invention.

FIG. 6 illustrates experimental results of images of Fe beads imaged through an agar solution with an imaging system according to an embodiment of the present invention.

FIGS. 7A-7B illustrate experimental results of imaging a rat heart muscle growth on a slide with a microscope (FIG. 7A) and with an imaging system according to an embodiment of the present invention (FIG. 7B).

FIGS. 8A-8D illustrate experimental results of imaging of blood veins in a chicken wing with a microscope (FIG. 8A) and with an imaging system according to an embodiment of the present invention (FIGS. 8B-8D).

FIG. 9 illustrates experimental results of imaging along a blood vein of a chicken wing with an imaging system according to an embodiment of the present invention.

FIG. 10 illustrates an experimental setup with an imaging system for imaging a rat brain according to an embodiment of the present invention.

FIG. 11 illustrates experimental results of imaging blood vessels inside a brain of a rat with an imaging system according to an embodiment of the invention.

### DETAILED DESCRIPTION OF EMBODIMENTS

The present invention proposes an imaging system that includes a multicore fiber (also referred to as a probe) containing tens of thousands of cores properly separated to avoid optical leakage between them even when bended (e.g. when navigated through a body). The structure of the probe allows performing resolution enhancement, i.e. super resolution based on shifts of an input tip of the probe. The structure of the probe also enables to perform field of view enhancement based on shifts of the input tip. Further, the optical cores of the multicore fiber act to transmit backwards a wave front and to generate an image, however one or more of the cores may also be used to illuminate the object itself or even to heat the object if illuminated with high photonic power density. Furthermore, illuminating the object with coherent light such as laser may allow extraction of 3D information by interference configuration near the detection plane in which not only the amplitude but also the phase of the reflected wave front can be estimated For example, an active reference beam at the detector array plane may be interfered with a wave front reflected by the object and transferred through the multicore fiber, thereby enabling to obtain phase information on the wave front reflected by the object. The phase information may enable to obtain 3D information on the object i.e. to build a profile of the object. In another embodiment, the interference configuration may be replaced by an holographic setup.

The probe allows realization of an optical operation equivalent to optical zooming, i.e. reducing the field of view and increasing the sampling resolution. This operation may be obtained by axially shifting an optical assembly (i.e. moving from the far field regime where we have large field of view and lower resolution into the near field approximation where we have good resolution and small field of view).

The cross section of the probe may be rectangular thereby allowing to coat two of its opposite faces with metals to realize electrical stimulation capability at its edge including heating/cooling or thermal sensing based upon the Pelletier effect when two different types of metals are used for the coating.

The proposed probe can be used for large variety of new biomedical applications in which its thin diameter allows noninvasive medical operability. The applications may involve navigation through blood artery, going through the tears holes into internal chambers in the nose and the head, performing navigation through lambs especially those of small children (having smaller channels) and performing prostate as well as womb related medical treatments.

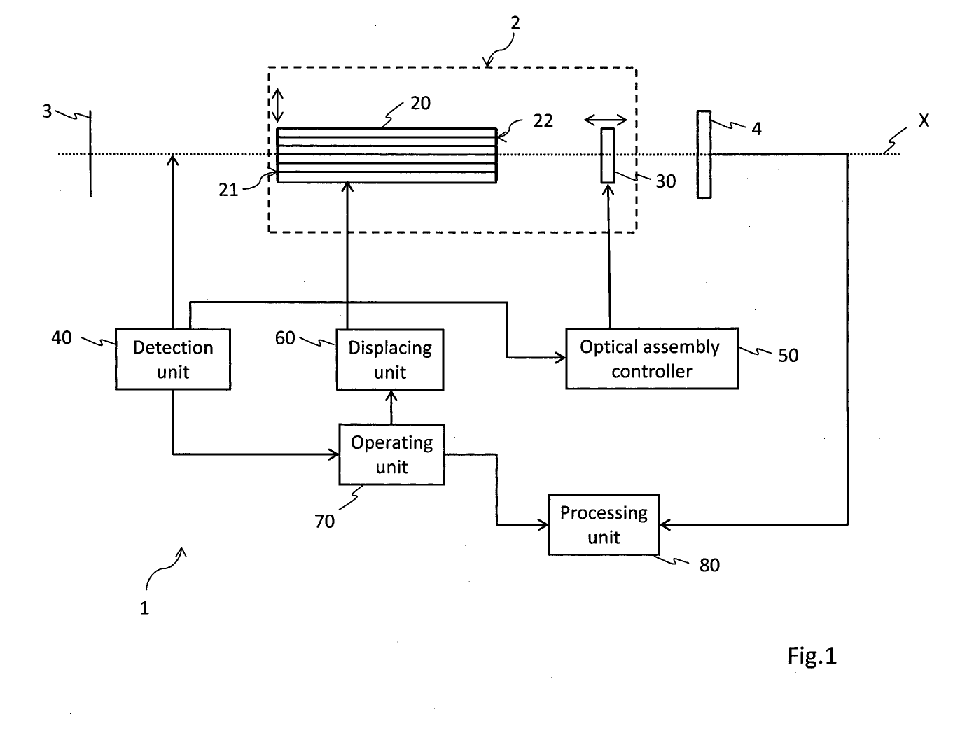

FIG. 1 illustrates generally an imaging system 1 according to an embodiment of the present invention. The imaging system 1 comprises an optical imaging unit 2 configured to form an image of an object 3 on a detector array 4 of the imaging system 1. The object 3 may be positioned far from the imaging unit 2 i.e. so that an optical wave front emitted by the object 3 and arriving to the imaging unit 2 may be considered as a plane wave front (far field approximation). The object 3 may alternatively be positioned close to the imaging unit 2 so that the previous far field approximation is not valid. Hereinafter, these two configurations are respectively referred to as far field and near field arrangements (or configuration, modes, etc.).

The optical imaging unit 2 defines an optical axis X and comprises a multicore fiber 20 (also referred to as a probe) and an optical assembly 30. The multicore fiber 20 may transfer light arriving from the object 3 to an input edge 21 of the multicore fiber 20 toward an output edge 22 of the multicore fiber 20. The optical assembly 30 may be configured to collect light at the output edge 22 of the multicore fiber 20 and to focus the light collected on the detector array 4. The optical assembly 30 may be configured to form images of the object 3 either positioned in the far field or in the near field of the imaging unit 2 i.e. relatively far or relatively close of the input edge 21. In an embodiment, the optical assembly 30 may be adaptable with regard to the position of the object 3 to be imaged on the detector array 4. In an embodiment, the optical assembly 30 may be an imaging lens positioned between the output edge 22 and the detector array 4. In said embodiment, the imaging lens may be longitudinally displaceable along the optical axis X so as to accommodate light from an object 3 positioned either in the far field or in the near field. In another embodiment, the optical assembly 30 may comprise an input lens positioned upstream of the input edge 21. The diameter of a core and the diameter of the multicore fiber 20 may be respectively referred to as d and D. The values of d and D are defined by fabrication and application related limitations. For example, D may be smaller than 300 μm in order to remain non invasive in certain medical applications. The value of d may be determined according to a desired spatial resolution. If D is equal to 300 μm and one wishes to have 100×100 pixels resolution it means that d may be about 3 μm. Generally, d may be larger than an optical wavelength of the light collected in order to allow coupling of light to the fiber with sufficient energetic efficiency.

The imaging system 1 may further comprise a detection unit 40, an optical assembly controller 50, a displacing unit 60, an operating unit 70 and a processing unit 80. The detection unit 40 may be configured to monitor the position of the object 3 with regard to the input edge 21. For example, the detection unit 40 may determine a longitudinal distance between the input edge 21 of the multicore fiber 20 and the object 3 to determine whether the object 3 is in near field or in far field. The detection unit 40 may comprise a detection communication utility to transmit data indicative of the longitudinal distance between the input edge 21 of the multicore fiber 20 and the object 3 to the optical assembly controller 50 and/or to operating unit 70. The optical assembly controller 50 may comprise a controller communication utility to communicate with the detection unit 40 so as to receive data from the detection unit 40 indicative of the longitudinal distance between the input edge 21 of the multicore fiber 20 and the object 3. The optical assembly controller 50 may be configured to adapt the optical assembly 30 based on said data so as to focus light emitted by the object 3 on the detector array 4. In other words, the optical assembly controller 50 may be configured to operate the optical assembly 30 so that the light output from the multicore fiber 20 forms an image on the detector array 4 according to a near field or far field configuration of the object 3. In the embodiment previously mentioned in which the optical assembly 30 is the imaging lens, the optical assembly adapting unit 40 may be configured to displace the imaging lens longitudinally along the optical axis X to focus light transferred by the multicore fiber 20 on the detector array 4. The optical assembly controller 50 may comprise a processor configured to process said data indicative of the longitudinal distance between the input edge 21 of the multicore fiber 20 and the object 3 so as to determine a configuration of the optical assembly 30 for imaging the object 3 according to conjugation relations. In the embodiment in which the optical assembly 30 is the imaging lens, since in respect to its imaging related property, the multicore fiber 20 may actually be regarded as if the input and output edges 21, 22 of the multicore fiber 20 act similarly to principle planes of an lens, the position of the optical assembly 30 may be determined according to the following relation:

$$\frac{1}{{U}_{1} + {U}_{2}} + \frac{1}{V} = \frac{1}{F} ,$$

wherein U₁ is the distance between the object 3 and the input edge 21 of the multicore fiber 20, U₂ is the distance between the output edge 22 of the multicore fiber 20, V is the distance between an optical center of optical assembly 30 and the detection array 4 and F is the focal length of the optical assembly 30.

The displacing unit 60 (or probe displacing unit) may be configured to shift the input edge 21 of the multicore fiber 20 relatively to the object 3. In an embodiment, the shift may be performed in a plane substantially perpendicular to the optical axis X (so-called “lateral shift”) in order to form a set of shifted images of the object 3 on the detector array 4. Alternatively, or preferably additionally, the shift may be performed along the optical axis (so-called “longitudinal shift”). This may be implemented by the same displacing unit moving the input edge 21 of the multicore fiber 20 along both axis, or additional displacement unit associated with a lens upstream or downstream of the probe.

The probe displacing unit 60 may comprise displacing communication utility configured to receive shifting amplitude instructions and/or shifting direction instructions from the operating unit 70 thereby enabling the operating unit 70 to operate the displacing unit 60. The operating unit 70 may further comprise a operating communication utility to communicate with the detection unit 40 so as to receive data from the detection unit 40 indicative of the longitudinal distance between the input edge 21 of the multicore fiber 20 and the object 3. The operating communication utility may further be configured to receive indications from an input utility (not shown) on whether a resolution or a field of view of the imaging is to be improved. The operating unit 70 may further comprise a shift controller configured to set a shifting amplitude to either a first amplitude inferior or equal to the diameter of a core of the multicore fiber 20 or a second amplitude superior or equal to the diameter of the multicore fiber 20. The setting of the amplitude may for example be based on the distance between the input edge 21 and the object 3 (i.e. a near field mode or far field mode) and on the input from a user received through said input utility.

The processing unit 80 may be connectable to the detector array 4 and configured to acquire and process the set of shifted images formed on the detector array 4 by interlacing said set of shifted images thereby obtaining a combined image of a better resolution or field of view. The operating unit 70 may provide the shifting amplitude set to the processing unit 80. For near field super resolving used in the present invention, the image processing of interlaced set of shifted images may be performed according to the spatial masking technique disclosed in references [9, 10]. For far field super resolving, the earlier image processing technique developed by the inventor and described in [11] may be used.

Therefore, the present system provides a compact and ergonomic solution simple to manufacture and able to provide images with a high resolution. Further, a user may rely on the operating unit to perform the shifting with a limited amount of manual operations.

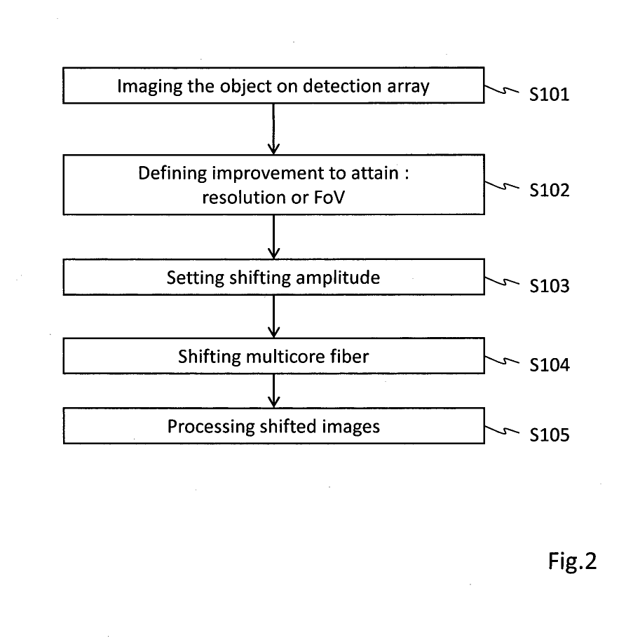

FIG. 2 illustrates generally steps of a method for imaging an object according to embodiments of the present invention. In a first step S101, an image of the object may be formed on a detector array using an optical imaging unit comprising a multicore fiber as an image guide and an optical assembly (for example an imaging lens) to focus light transferred by the multicore fiber on the detector array. The positioning of the optical assembly may be performed based on the position of the object i.e. based on a near field or far field arrangement. The image may further be displayed on a display unit. In a second step S102, a user may define an improvement to attain in the image between resolution improvement and field of view improvement, based for example on observation of the displayed image. In a third step S103, based on the improvement defined and on whether the object is positioned in the near field or in the far field of the imaging unit, a shifting amplitude may be set between a first amplitude inferior or equal to the diameter of a core of the multicore fiber and a second amplitude superior or equal to the diameter of the multicore fiber.

In the near field mode and to improve resolution, given a predetermined super resolving factor K (K being an integer) to be achieved, the shifting amplitude may be set to the first amplitude. The first amplitude may be determined by the following relation:

A₁=d/K,

wherein d is the diameter of the core of the fiber. Further, the number of shifts performed may be equal to the super resolving factor.

In the far field mode and to improve resolution, given the predetermined super resolution factor, the shifting amplitude may be set to the second amplitude. The second amplitude may be determined by the following relation:

A₂=D,

wherein D is the diameter of the multicore fiber. Further, the number of shifts may be equal to the super resolving factor.

In order to increase the field of view (and not the resolution) in near field, the shifting may be performed with the second shifting amplitude A₂. Particularly, in order to increase the field of view by a factor of K one may perform K shifts with the second shifting amplitude A₂. In the far field, in order to increase the field of view by a factor of K one may perform K shifts with the first shifting amplitude A₁.

The position of the object may be detected in order to determine if the object is positioned in the near field or in the far field of the imaging unit. For example, the longitudinal distance between an input edge of the multicore fiber and the object may be detected by a sensor. In an embodiment, the shifting amplitude is set to the second amplitude when (a) the object is in the far field and the resolution is to be improved, and/or (b) the object is in the near field and the field of view to be improved. In an embodiment, the shifting amplitude is set to the first amplitude when (c) the object is in the near field and the resolution is to be improved, and/or (d) the object is in the far field and the field of view is to be improved. Therefore, the same shifting amplitude may be used for different purposes i.e. enhancing resolution or field of view when imaging an object in different modes i.e. in far field and near field mode. In a fourth step S104, the multicore fiber input edge may be shifted of said shifting amplitude in order to obtain a set of shifted images. In a fifth step S105, the set of shifted images may be processed to obtain a combined image which attains the improvement defined i.e. a better resolution or field of view than the image obtained in step S101 (also referred to as the original image).

The implementation of the processing of the set of shifted images and the setting of the shifting amplitude based on the relative position of the object with regard to the multicore fiber and on the imaging improvement to attain in the original image may be better understood considering the following:

Any imaging system has limited capability to discriminate between two spatially adjacent features. The physical factors that limit this capability can be divided into two types. The first type is related to the effect of diffraction of light being propagated from the object towards the imaging sensor [8]. The resolution limit due to diffraction as it is obtained in the image plane equals to:

δₓ=1.22λF_{#}

where λ is the optical wavelength and F_{#} denotes the F number which is the ratio between the focal length and the diameter of the imaging lens.

The second type is related to the geometry of the detection array [8, 9]. The geometrical limitation can be divided into two kinds of limitations. The first is related to the pitch of the sampling pixels i.e. the distance between two adjacent pixels. This distance determines, according to the Nyquist sampling theorem, the maximal spatial frequency that can be recovered due to spectral aliasing (generated when signals are under sampled in the space domain):

δ_{pitch}=1/2υₘₐₓ=1/BW

where δ_{pitch} is the pitch between adjacent pixels, υₘₐₓ is the maximal spatial frequency that may be recovered and BW is the bandwidth of the spectrum of the sampled image. The second kind is related to the shape of each pixel and to the fact that each pixel is not a delta function and thus it realizes a non ideal spatial sampling.

In fact, the type of resolution reduction that is being imposed by the multi core probe depends on the distance between the edge of the probe and the object (previously denoted U₁).

Diffraction resolution reduction is obtained when the input plane of the probe is relatively away from the object (far field approximation) and then the light distribution on this plane resembles the light distribution over the imaging lens aperture. In that case the diameter of the fiber D sets the maximal spatial frequency transmitted by the fiber and therefore also the spatial resolution obtainable in the image plane:

δₓ=λV/D

and the fact that there are multiple cores is equivalent to sampling in the Fourier plane which means replication in the image plane yielding limiting restriction over the obtainable field of view:

Δₓ=λV/d

where Δₓ is the obtainable field of view in the image plane and d is the pitch between two adjacent cores in the multi core probe.

The geometrical limitation is obtained when the distance between the fiber and the object (U₁) is relatively small (near field approximation) and then the field of view is limited by the diameter of the fiber D while the pitch between two cores d determine the spatial sampling resolution:

Δₓ=MD and δₓ=Md

where M is the demagnification factor of the proposed imaging system and it equals to:

M=V/(U₁+U₂)

The imaging method of the present invention selectively overcomes the geometrical limitation or the diffraction limitation based on detecting whether the object is in near field or in far field and on accordingly setting appropriate shifting amplitude to obtain a set of shifted images thereby enabling to conduct super resolution processing. In super resolution the idea is to encode the spatial information that could not be imaged with the optical system into some other domain. Transmit it through the system and to decode it [8]. The most common domain to do so is the time domain.

Therefore, a way for obtaining resolution improvement in the proposed configuration can be as follows: in the case of far field arrangement when the limiting factor is related to diffraction, the fiber itself can be shifted in time. This time scanning operation will be equivalent to generation of a synthetically increased aperture similar to what happens in synthetic aperture radars (SAR). In this scanning operation the resolution improvement factor is proportional to the ratio between the scanned region and the diameter of the probe D. If instead of super resolution one wishes to increase the imaging field of view, the probe needs to be shifted at amplitude of less than d in order to generate over sampling of spectrum domain by its multiple cores. In this case a set of images are captured while each is obtained after performing a shift of sub core distance. Then, all the images are interlaced together accordingly to generate effective sub core sampling. In the case of near field approximation, temporal scanning once again can improve the resolving capability as described in Refs. [9,10]. In this case the shift is limited by the size of d. Once again a set of images are captured while each is obtained after performing a shift of sub core distance. Then, all the images are interlaced together accordingly to generate effective sub core sampling. In case that instead of resolution improvement one wishes to obtain an increase in the imaging field of view, the probe can again perform scanning but this time at larger amplitude. The field of view enlargement is proportional to the ratio between the shift amplitude and the diameter of the probe D.

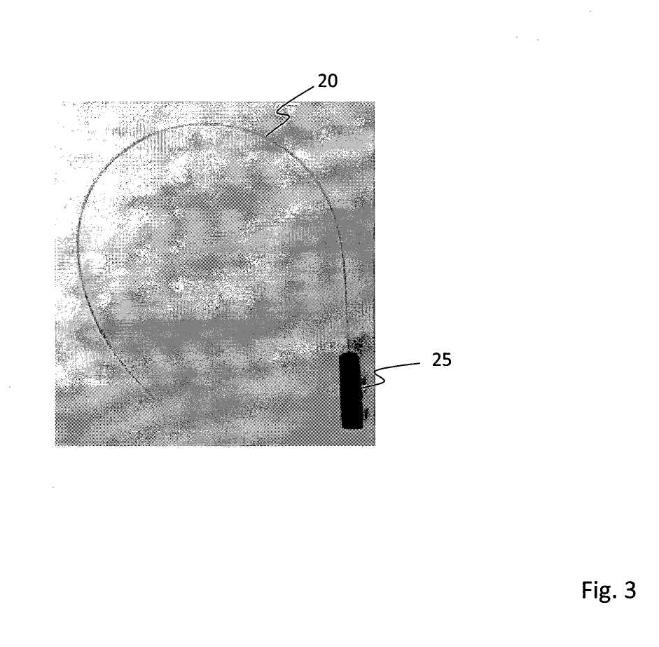

FIG. 3 illustrates a multicore fiber 20 of an imaging system according to an embodiment of the present invention. The multicore fiber 20 may be held at one edge by a holder 25. The multicore fiber 20 may have a large number of optical cores to be sufficient to allow high resolution imaging functionality where the high resolution imaging is performed using the previously described method. The number of fabricated cores in this fiber is about 5,000-10,000. This micro probe is made out of polymers, while the core is made out of PS (Polystyrene) and the cladding is made out of Poly(methyl methacrylate) (PMMA). The refractive indexes of the PS and the PMMA at a wavelength of 632 nm are 1.59 and 1.49, respectively.

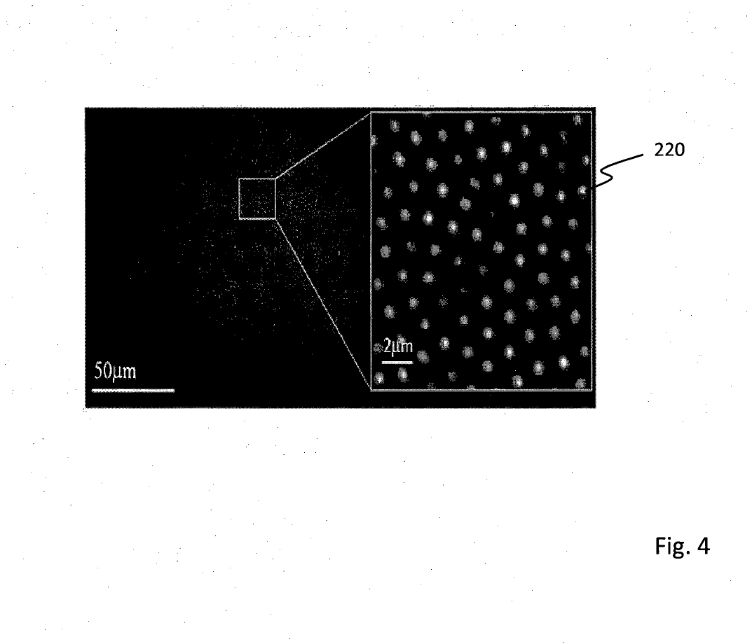

FIG. 4 presents the multi cores 220 visible from the output edge of the probe transferring light emitted by an object. The probe presented in FIG. 4 has about 5,000 cores but the actual spatial resolution even with this number of cores might be larger due to super resolution processing that is to be elaborated according to the method previously described. In the figure one may see that thousands of individual light channels transmitting spatial information at wavelength of 630 nm. Each channel may basically be a different pixel in the constructed image. As previously mentioned, the fact that each pixel may be transmitted individually and the endoscope may basically be a multi core fiber rather than a multi mode fiber allows generating an image that is insensitive to bending of the fiber. This feature enables the multicore fiber to be bent and to perform for example an endoscopy procedure.

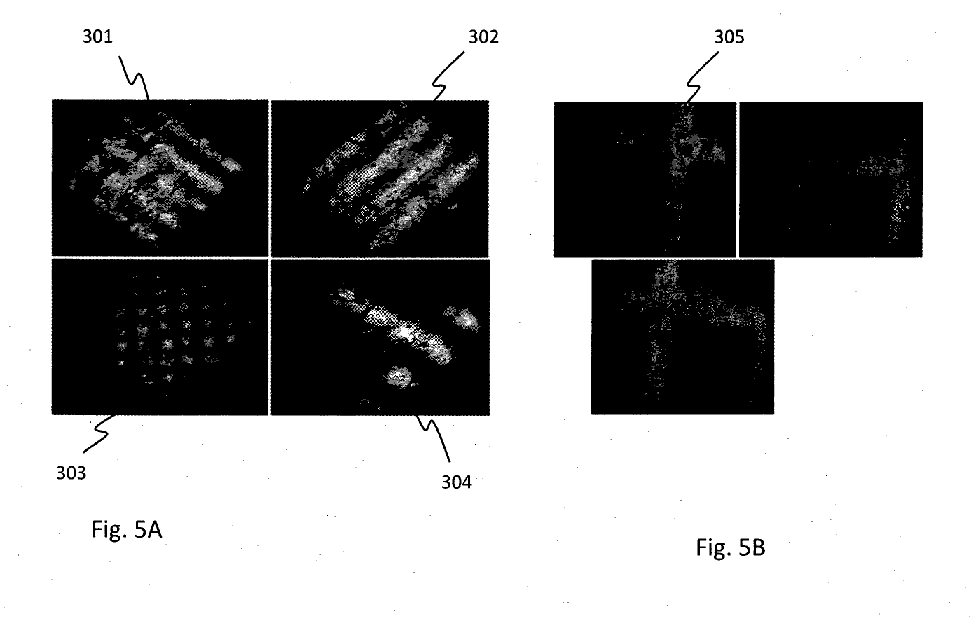

FIG. 5A and FIG. 5B present experimental results of images acquired by an imaging system according to an embodiment of the invention. FIG. 5A presents high resolution target imaging with the micro probe of FIG. 3. The presented experimental results include images that were transmitted backwards by the multicore fiber and imaged on a detector array (CCD camera). The imaged object was a resolution target while the presented images include in the upper line from left to right: an object 301 with rotated black vertical lines and an object 302 with rotated black vertical lines rotated at 90 degrees with respect to object 301. In the second row form left to right we have an object 303 with small black rectangles and then an object 304 with large black lines and black rectangle appearing in the left side of the backwards transmitted image. FIG. 5B shows three shifted images of a square like shape resolution target 305 imaged at different locations i.e. by shifting the input edge of the multicore fiber with regard to the square like shape resolution target 305 using an imaging system according to an embodiment of the present invention including the multicore fiber shown on FIG. 3. This figure demonstrates that although the multicore fiber (micro probe) is very thin in its diameter, a large field of view may be obtained by proper cascading the acquired shifted images.

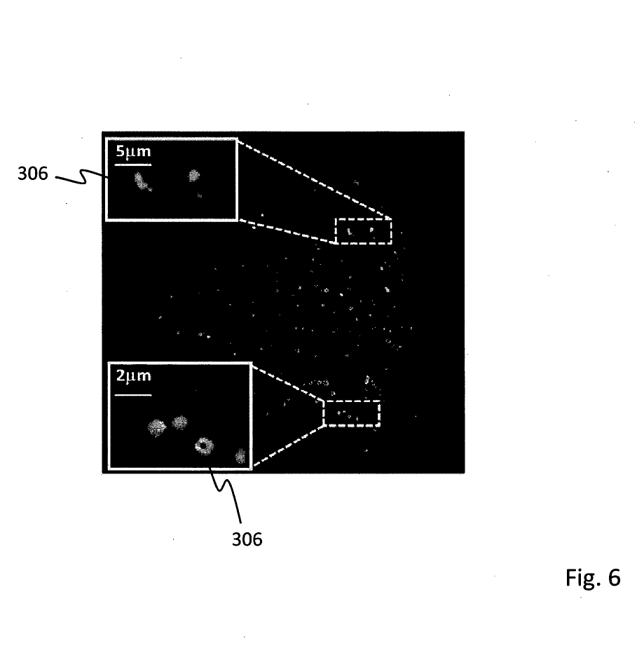

FIG. 6 presents experimental results of images of Fe beads 306 having diameter of around 1 μm imaged through an agar solution. In order to further demonstrate the high spatial discrimination the experiment is repeated. The imaging of the Fe micro beads 306 is performed through agar solution in order to show that imaging through biological like medium is possible and to demonstrate the high spatial resolution of the fabricated prototype allowing spatial separation between sub micron features. The experimental results presented in FIG. 6 were performed by using an imaging system according to an embodiment of the invention including the multicore fiber shown on FIG. 3.

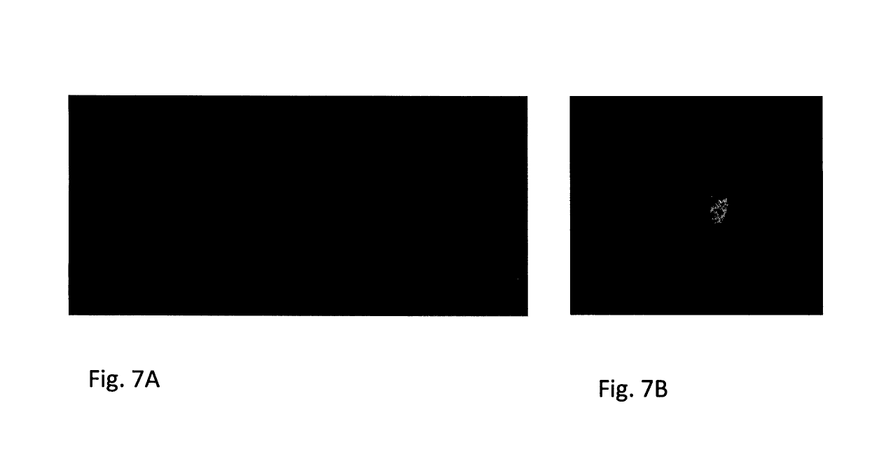

FIG. 7A and FIG. 7B present imaging of rat heart muscle growth on a slide. FIG. 7A shows a top view microscope image of the rat heart muscle, while FIG. 7B shows these cells (shown in the inset image of FIG. 7A) imaged with tan imaging system according to an embodiment of the present invention including the multicore fiber shown on FIG. 3. Cell culture and sample preparation for measurements was as follows: Rat cardiac myocytes were isolated. Briefly, hearts from newborn rats were rinsed in phosphate buffered saline (PBS), cut into small pieces and incubated with a solution of proteolytic enzymes-RDB (Biological Institute, Ness-Ziona, Israel). Separated cells were suspended in Dulbecco's Modified Eagle's Medium (DMEM) containing 10% inactivated horse serum (Biological Industries, Kibbutz Beit Haemek, Israel) and 2% chick embryo extract, and centrifuged at 300×g for 5 min. Precipitant (cell pellet) was resuspended in growth medium and placed in culture dishes on collagen/gelatin coated cover-glasses. On day 4, cells were treated with 0.5-5 μM of DOX for 18 hours and then with drug-free growth medium for an additional 24 hours. Cell samples were grown on microscope cover slips and imaged by an imaging system according to an embodiment of the present invention including the multicore fiber shown on FIG. 3.

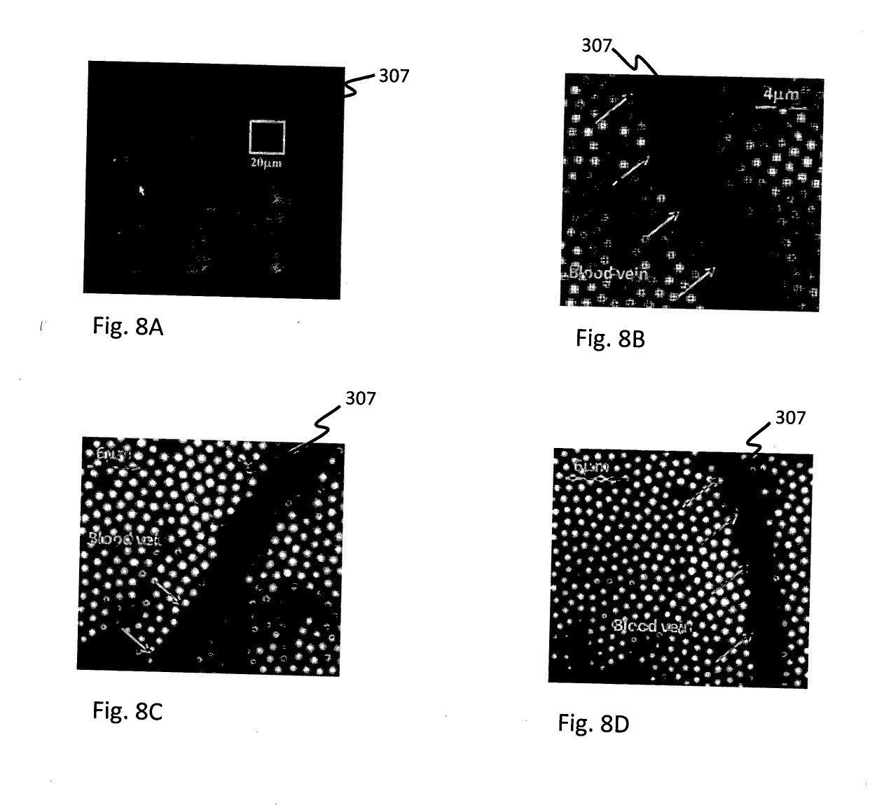

FIG. 8A-8D present imaging of blood veins inside a chicken wing. FIG. 8A shows a top view microscope image of veins 307 of the chicken wing area, while FIGS. 8B-8D show the veins 307 (indicated by solid arrows) imaged with an imaging system according to an embodiment of the invention including the multicore fiber shown on FIG. 3.

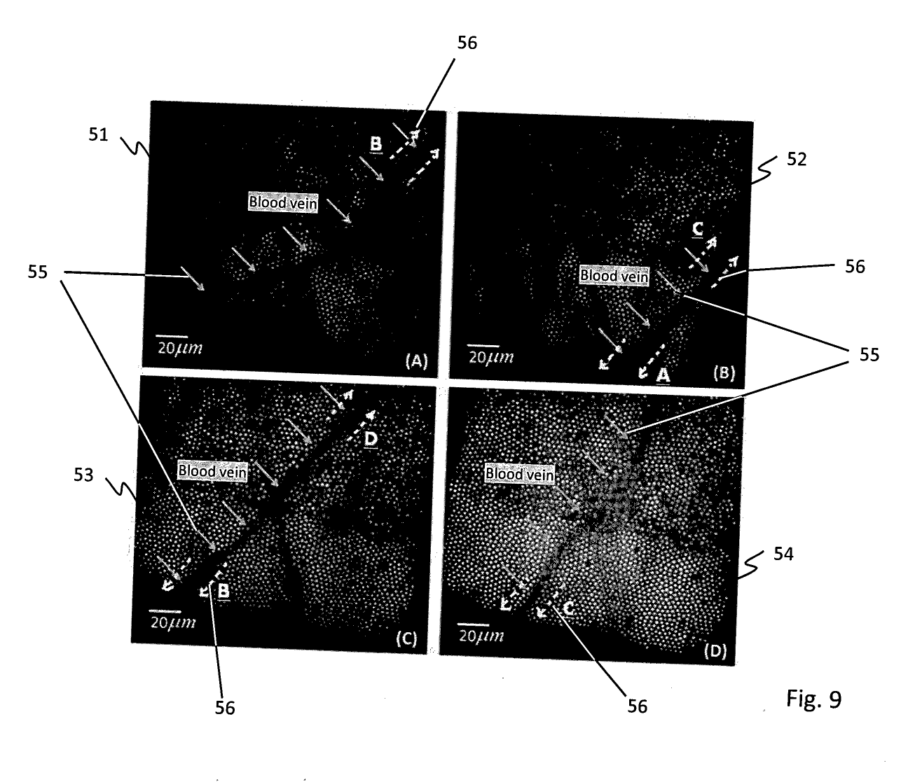

FIG. 9 illustrates experimental results of imaging along a chicken wing with an imaging system according to an embodiment of the present invention including the multicore fiber shown on FIG. 3. Images 51-54 show shifted images of a blood vein (shown with solid arrows) in the chicken wing. Images 51-54 have been taken by shifting the input edge of the multicore fiber. Although, the multicore fiber (micro probe) diameter is equal to 200 μm, a construction of a large image can be obtained with real time image processing. This can be done by calculating the shift (or relative movement) of the probe displacing unit (in the present example, a multi axis platform where the micro probe is located) and converting it to the image movement. Each one of the images 51-54 shows different location of the micro probe along the imaged blood vein. In fact, from the visualization point of view, the images are individually shown instead of being cascaded into a single image. The solid arrows 55 indicate the blood vein, while the dashed arrows as well as the labeling letter indicate the cascading point between the images presented in FIG. 9 The four images 51-54 correspond to the references A, B, C and D shown on FIG. 9 and in each of images 51-54, the dashed lines 56 indicate in which position in respect to a given image the other images appear. So each of the four images is sectioned in a larger field of view. By proper cascading the images a full length of the examined area of interest (e.g. blood vein) can be obtained. Such cascading may also be referred to as a mosaicing or an image processing to improve field of view.

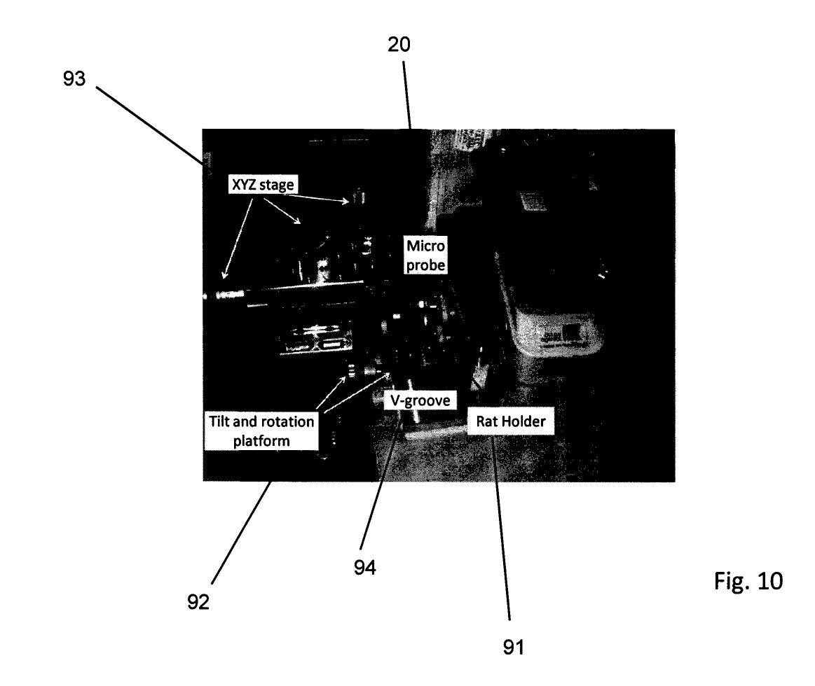

FIG. 10 shows the experimental setup used for imaging from inside a brain of a rat. The setup includes a first module and a second module. The first module comprises a special rat holder 91 that is used to hold the rat during the surgery and the experiments processes and the second module is related to a probe displacing unit (platform) comprising a tilt and rotation platform 92, an XYZ stage 93 and a V groove 94 that enables accurate navigation of the micro probe 20 inside the examined area. Navigating the multicore fiber (micro probe) inside the brain tissue may be done by using the probe displacing unit which provides five axis positioning stage through the XYZ stage 93 and the tilt and rotation platform 92. On top of the multi axis stage the V-groove is configured as a bare fiber holder which allows locating the micro probe at an examined area.

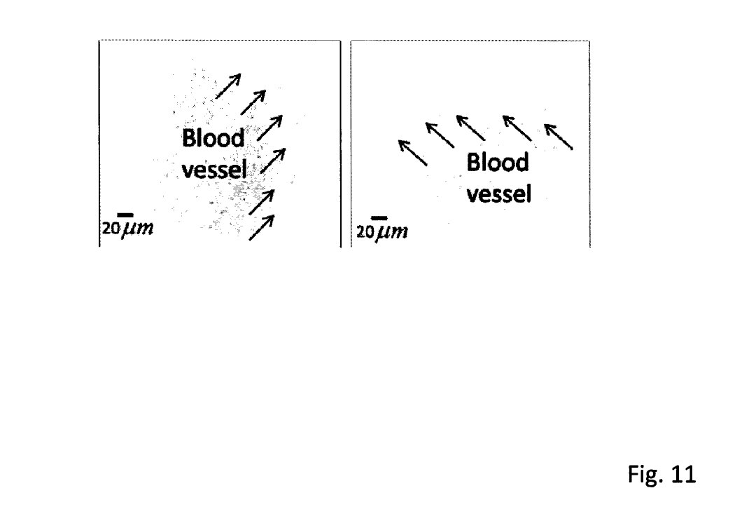

FIG. 11 is a picture with inversed colors showing blood veins (indicated by solid arrows) of rat brain imaged by an imaging system according to the present invention including the multicore fiber shown on FIG. 3 and using the experimental setup described on FIG. 10. Imaging is performed by penetrating the micro probe inside the brain tissue. In brief the surgical procedure was as follows: the rat has been placed in a cage where isofleurane is injected through a vaporizior. Right after, the rat is anaesthetised using injection of urethane and positioned at a special rat head holder. Then an approximately 7 mm diameter hole is drilled above the barrel cortex of the rat, which is identified by the anatomical coordinates. The dura is gently removed to perform the experiments procedure. Note that the proposed probe that is being realized is very thin in its diameter in comparison to commonly used endoscope, while it does not provide only spatial resolution that is as good but also enable both penetration inside the examined area and minimal invasive damage as well.

The above examples and description have of course been provided only for the purpose of illustration, and are not intended to limit the invention in any way. As will be appreciated by the skilled person, the invention can be carried out in a great variety of ways, employing more than one technique from those described above, all without exceeding the scope of the invention.

1. A system for imaging an object comprising:
an optical imaging unit for imaging an object on a detection array, the optical imaging unit defining an optical axis and comprising a multicore fiber configured to collect light from the object at an input edge of the multicore fiber and transfer collected light to an output edge of the multicore fiber;
a displacing unit configured to shift the input edge of the multicore fiber relatively to the object in a plane substantially perpendicular to the optical axis to obtain a set of shifted images of the object; and
an operating unit configured to operate the displacing unit by setting a shifting amplitude to either a first amplitude inferior or equal to the diameter of a core of the multicore fiber or a second amplitude superior or equal to the diameter of the multicore fiber.

2. The system according to claim 1, wherein the optical imaging unit comprises an optical assembly configured to collect light from the output edge of the multicore fiber and form an image of the object on the detection array.

3. The system according to claim 1, wherein the optical imaging unit is configured for selectively operating in either one of a near field and far field imaging modes.

4. The system according to claim 3, wherein the optical imaging unit comprises a lens unit arranged upstream of the input edge of the multicore fiber with respect to a direction of light propagation through the system, said lens unit being displaceable along the optical axis with respect to the object.

5. The system according to claim 1, comprising a processing unit connectable to the detection array and configured to receive and process data indicative of the set of shifted images for obtaining a combined image of the object by interlacing one or more of said shifted images, said combined image having the improved resolution and/or field of view.

6. The system according to claim 3, further comprising a detection unit configured to monitor a distance between the object and the input edge of the multicore fiber to thereby enable the operation of the optical imaging unit in either one of the near field and far field imaging modes.

7. The system according to claim 1, further comprising a display unit for displaying at least one of the images.

8. The system according to claim 1, wherein the operating unit comprises an input utility configured to receive input from a user defining whether the field of view or resolution of the imaging is to be improved.

9. The system according to claim 1, wherein the operating unit further comprises:
a communication utility to communicate with the detection unit; and
a shift controller for setting the shifting amplitude based on a distance between the object and the input edge of the multicore fiber and on whether a field of view or a resolution of the original image is to be improved.

10. The system of claim 9, wherein the operating unit comprises a communication utility to communicate with the input unit.

11. The system according to claim 1, wherein the multicore fiber is either a fiber bundle or a photonic crystal.

12. The system according to claim 1, wherein the multicore fiber has a polygonal cross section defining two opposite substantially parallel facets.

13. The system according to claim 12, wherein the cross section of the multicore fiber is rectangular.

14. The system according to claim 12, further comprising electrodes located on said opposite facets of the multicore fiber to carry out at least one of electrical stimulation and sensing temperature using Peltier effect.

15. The system according to claim 1, further comprising:
a coherent light source configured to illuminate the object and provide a reference wave front; and
an holographic or interferometric setup configured to provide interference between the reference wave front and a reflected wave front reflected by the object and transferred by the multicore fiber, thereby providing phase information on light reflected by the object.

16. A method for imaging an object comprising:
transferring light coming from the object through a multicore fiber having an input edge and an output edge;
imaging the object on a detection array by collecting light from the output edge of the multicore fiber;
setting a shifting amplitude for multicore fiber, such that the shifting amplitude is either a first amplitude inferior or equal to the diameter of a core of the multicore fiber or a second amplitude superior or equal to the diameter of the multicore fiber, to enable improvement of either resolution or the field of view of imaging;
shifting the input edge of the multicore fiber in a plane substantially perpendicular to an axis of light propagation from the object to the detection array, using said shifting amplitude, thereby obtaining a set of shifted images of the object; and
processing said set of shifted images in order to obtain a combined image of the object by interlacing said shifted images for improving the resolution or field of view.

17. The method according to claim 16, comprising selectively imaging the object in either one of a near field and far field imaging modes.

18. The method according to claim 17, comprising moving a lens unit, in front of the input edge of the multicore fiber, along said axis of light propagation, with respect to the object.

19. The system according to claim 13, further comprising electrodes located on said opposite facets of the multicore fiber to carry out at least one of electrical stimulation and sensing temperature using Peltier effect.
